(19) United States
(12) Patent Application Publication
(10) Pub. No.: US 2004/0133255 A1
(43) Pub. Date: Jul. 8, 2004

(54) Body temperature control device and body temperature controller using the control device

(75) Inventors: Katsuyuki Miyasaka, Tokyo (JP); Kazufuku Nitta, Saitama-ken (JP)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Metran Co., Ltd.

(21) Appl. No.: 10/737,945

(22) Filed: Dec. 17, 2003

Related U.S. Application Data
(63) Continuation of application No. PCT/JP02/06006, filed on Jun. 17, 2002.

(30) Foreign Application Priority Data
Jun. 25, 2001 (JP) 2001-191746

(51) Int. Cl. A61F 7/00

(52) U.S. Cl. 607/105

(57) ABSTRACT

The present invention provides a body temperature control device capable of efficiently controlling body temperature, and a temperature controller using this device.

The body temperature control device of the present invention is brought into contact with a patient. Then, a fluid used for temperature control is fed into the inside of a hollow fiber by a body temperature controller. A heat transfer medium passing through the hollow fiber, performs heat exchange with a particular part of the body of the patient, to control the temperature of that part of the patient's body. The body temperature control device using the hollow tube can be made highly flexible, which means that it is possible have a wide contact area with the body, which improves the efficiency of heat exchange.

### CROSS-REFERENCE TO RELATED APPLICATION

This application is a continuation of prior Application No. PCT/JP02/006006, filed Jun. 17, 2002, priority from the filing date of which is hereby claimed under 35 U.S.C. § 120.

### FIELD OF THE INVENTION

The present invention relates to a body temperature control device, and to a body temperature controller using the control device.

### BACKGROUND OF THE INVENTION

In the course of medical treatment, it is sometimes necessary to control the body temperature of a patient. The following are examples of such situations.

1) when lowering the body temperature to about 50 in order to safely stop the heart beating during a cardiotomy etc.;

2) when raising the body temperature slightly because new born and infant operations are susceptible to lowering of body temperature;

3) when slightly cooling the body to around 350, in order to prevent rise in body temperature after an operation or for trauma patients;

4) when slightly lowering body temperature to reduce a fever accompanying an infection or a fever accompanying vasoconstriction caused by reduced cardiac output etc.

As a body temperature controller currently used in these situations, it is well known to circulate hot or cold water through a mat, and to make a patient lie down on the mat. However, with this technique, contact efficiency between the heat control mat and the patient is poor, and there is a problem of poor energy efficiency in body temperature control.

The present invention has been conceived based on this situation, and an object of the invention is to provide a body temperature control device and a body temperature controller using the control device capable of efficient body temperature control.

### SUMMARY OF THE INVENTION

A body temperature control device of the present invention is constructed using hollow fibers, and a heat transfer medium is provided inside the hollow fibers.

The heat transfer medium of the body temperature control device is, for example, a fluid.

The body temperature control device can be formed in a substantially sheet shape.

The body temperature control device can also be flexible.

The body temperature control device can also be formed in a hat shape.

With the body temperature control device it is also possible to incorporate the hollow fibers as one of either warp fiber or weft fiber.

With the body temperature control device it is also possible to have elastic fastening between the hollow fibers and the other of the warp or weft fiber.

It is possible to form concave or convex sections on an inner surface of the hollow fibers of the body temperature control device.

It is also possible for the hollow fibers of the body temperature control device to be hydrophobic.

It is also possible to attach a heat insulating material to a surface of the body temperature control device opposite to a user.

A body temperature controller of the present invention comprises the body temperature control device and a controller body for delivering heat transfer medium to the inside of hollow fibers of the body temperature control device.

A body temperature control method of the present invention comprises the steps of bringing the body temperature control device close to a patient, and supplying heat transfer medium to the body temperature control device.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an explanatory drawing for describing the overall schematic structure of a body temperature controller of a first embodiment of the present invention.

FIG. 2 is a schematic cross sectional drawing of essential parts of a body temperature control device of the first embodiment of the present invention.

FIG. 3 is a cross sectional drawing showing one example of a hollow fiber used in the first embodiment of the present invention.

FIG. 4 is an enlarged schematic cross sectional view of essential parts of a body temperature control device of a second embodiment of the present invention.

FIG. 5 is a schematic cross sectional view of a usage state of a body temperature control device of a third embodiment of the present invention.

### DETAILED DESCRIPTION OF THE PREFERRED EMBODIMENT

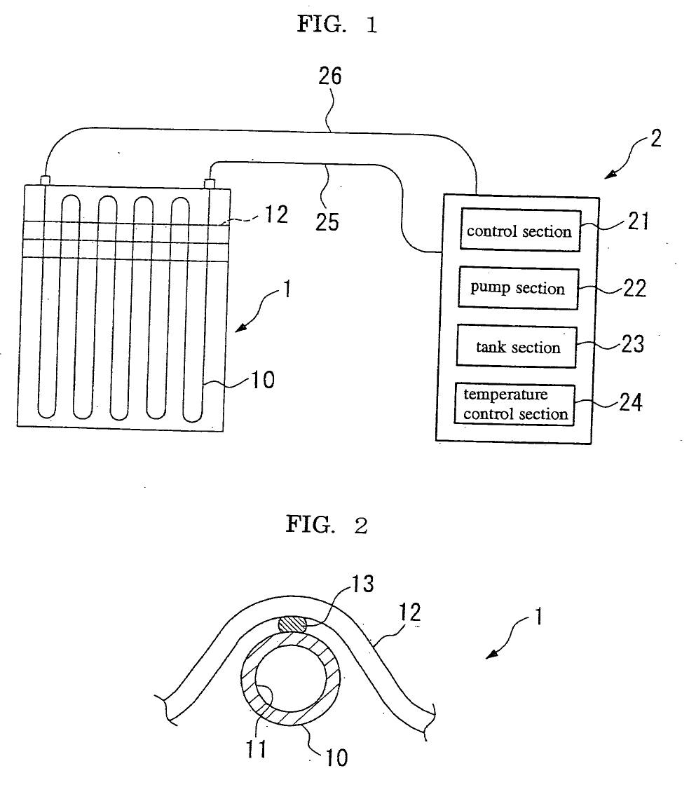

A body temperature control device of a first embodiment of the present invention and a body temperature controller using the body temperature control device will be described in the following. The body temperature controller is shown in FIG. 1. This body temperature controller is mainly composed of a body temperature control device 1 and a controller body 2.

The body temperature control device 1 is constructed using hollow fiber. The hollow fiber 10 is filled with a heat transfer medium (a medium for heat exchange, in this body a fluid, particularly water) from the controller body 2. The controller body 2 will be described later. The body temperature control device 1 is formed having an overall sheet shape. Also, the body temperature control device 1 is flexible overall.

In more detail, the body temperature control device 1 is composed of hollow fiber 10 as warp fibers, incorporated using arbitrary weft fibers 12. In FIG. 1, only some of the weft fibers 12 are shown. Since the hollow fiber 10 itself is flexible, with this structure it is possible to impart flexibility to the body temperature control device 1 itself. Also, as shown in FIG. 2, by flexibly connecting between the hollow fiber 10 and the weft fibers 12 using an elastic contact section 13 (refer to FIG. 2), it is possible to improve the flexibility of the body temperature control device 1.

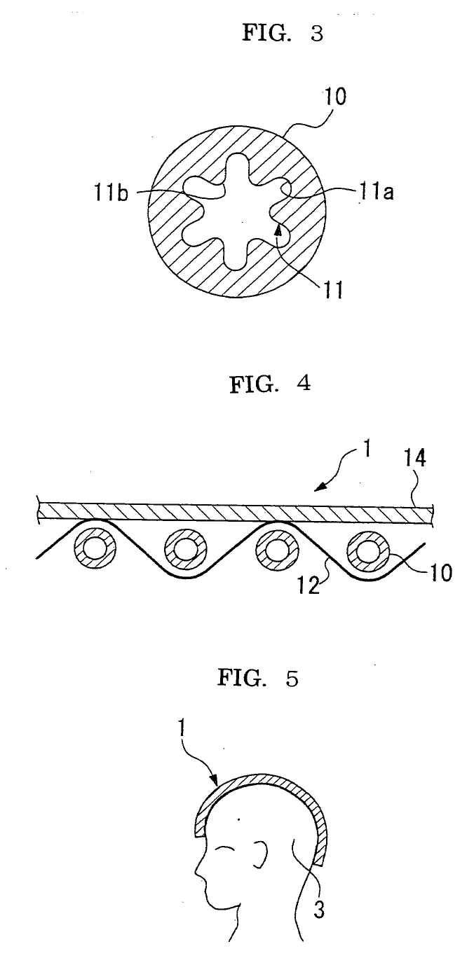

As the hollow fiber 10, it is possible to use ordinary fiber. Besides normal fiber, it is also possible, for example, to form concave sections 11a and convex sections 11b on an inner surface 11 of the hollow fiber 10, as shown in FIG. 3. If this is done, it is possible to ensure a flow path for the heat transfer medium even if the hollow fiber 10 is compressed, and it is possible to maintain a body temperature control function.

The shape of the body temperature control device 1 can be any shape, depending on the method of use, such as wrapping around a place where body temperature is to be controlled, being worn, covered with or laid upon.

The controller body 2 feeds a heat transfer medium for heat exchange (water in this embodiment) to the inside of the hollow fiber 10 of the body temperature control device 1. The controller body 2 comprises a control section 21, a pump section 22, a tank section 23, a temperature control section 24, an up pipe 25 and a down pipe 26. The control section 21 detects temperature of the heat transfer medium flowing in the down pipe 26 and performs control based on a difference between the detected temperature and a set temperature so that heat transfer at an appropriate flow rate and temperature is fed to the up pipe 25. The control section 21 can be easily configured using a suitable sensor and a microcomputer and software, and so detailed description will be omitted.

The pump section 22 sends out heat transfer medium from the tank section 23 to the up pipe 25 in response to an instruction from the control section 21. Obviously, it is also possible to send out heat transfer medium from the tank section 23 to the hollow fiber 10 by subjecting the down pipe 26 to negative pressure.

The tank section 23 holds a required amount of heat transfer medium. The tank section 23 is preferably configured having a heat retaining function for the heat transfer fluid, using a heat insulating material or any other appropriate means.

The temperature control section 24 comprises structures for appropriately regulating temperature of heat transfer medium fed from the up pipe 25 to the body temperature control device 1. Specifically, a heater if it is for heating, or a cooler if it is for cooling. The temperature control section 24 generally controls temperature of the heat transfer medium stored in the tank section 23. Operation of the temperature control section 24 is controlled by the control section 21.

The up pipe 25 is connected to one end of the hollow fiber 10 by a well known method (for example, a potting method), making it possible to feed heat transfer medium into the inside of the hollow fiber 10. Similarly, the down pipe 26 is connected to the other end of the hollow fiber 10 making it possible to recover heat transfer medium that has passed through the inside of the hollow fiber 10.

Next, the method of using the body temperature controller of this embodiment having the above described structure will be described. First of all, the body temperature control device 1 is brought into contact with the body of a patient by wrapping around a necessary place, or being worn, covered with or laid upon. Then, heat transfer medium for body temperature control is fed to the up pipe 25 by the body temperature controller 2. The fed heat transfer medium passes through the inside of the hollow fiber 10. The heat transfer medium passing through the hollow fiber 10 carries out heat exchange with a particular part of the body of a patient, and control the body temperature at that part of the body. As a result, according to the body temperature control device 1 and body temperature controller of this embodiment, it is possible to control body temperature by feeding heat transfer medium whose temperature has been appropriately controlled into the hollow fiber 10. For example, if the heat transfer medium is made low temperature, it is possible to lower body temperature, while if the heat transfer medium is made high temperature it is possible to raise body temperature.

Also, with this embodiment, it is also possible to improve heat transfer efficiency by increasing the heat transfer fluid flow rate. In this case, it is possible to use a smaller amount of heat transfer medium, enabling reduction in size and weight of the device.

Also with this embodiment, since the body temperature control device 1 is mainly constituted by the hollow fiber 10, there is the advantage that it is easy to impart high flexibility to the body temperature control device 1.

With this embodiment, since the body temperature control device 1 is flexible, it is possible to have a large contact area between the body temperature control device 1 and the body, and there is the advantage that body temperature control efficiency is improved.

It is also possible to make the hollow fiber 10 hydrophobic. In this case, since the hollow fiber 10 does not absorb water, there is no dehydration of internal organs even if internal organs are packed using the body temperature control device 1. It is therefore possible to directly cool internal organs. Conventionally, cooling of internal organs in organ transplants is widely carried out by placing internal organs directly into a cooling medium. However, with this method it is difficult to finely control the cooling temperature of the internal organs to an appropriate temperature. With this embodiment, it is possible to appropriately control the temperature of the internal organs using the body temperature control device 1. Also, in the case of the hollow fiber 10 being made hydrophobic, a liquid (for example water) as the heat transfer medium used evaporates, humidifying around the hollow fiber 10. If this is done, there is the advantage that it is possible to further improve heat transfer efficiency between the body and the heat transfer medium. Also, it is difficult for microorganisms to breed on the surface of the hydrophobic hollow fiber 10, which means that there is the advantage that it is made easier to keep the body temperature control device 1 in a hygienic state. In this specification, the word patient is intended to include parts of the human body (for example, internal organs).

It is also preferable to use an extremely flexible material as the material for the hollow fiber 10, weft fibers 12, and contact section 13, and also a material or construction that does not harden when heated or cooled. In this way, it is possible to further improve the contact area between the body temperature control device 1 and the body at the time of body temperature control, and it is possible to further improve body temperature control efficiency.

Next, a body temperature control device 1 of a second embodiment of the present invention will be described based on FIG. 4. With this body temperature control device 1, a heat insulating material 14 is attached to a side of the body temperature control device 1 opposite to the patient side. As the heat insulating material 14, there is fabric of a woolen material for example, but this is not limiting. It can also be a fabric or a non-woven fabric, but again, is not limited to this. As a method of attaching the heat insulating material 14 to the body temperature control device 1, any method can be used. For example, when the body temperature control device 1 is woven using weft fibers 12, it is possible to use a method of weaving the insulating material 14 together. Alternatively, it is also possible to use a method of fixing a separately manufactured heat insulating material 14 to one surface of the body temperature control device 1.

According to the body temperature control device 1 of the second embodiment, it is possible to reduce heat exchange between heat transfer medium passing through the hollow fiber 10 and the outside using the heat insulating material 14. In this way, it is possible to reduce energy loss used in temperature control, and there is the advantage that it is possible to improve temperature control efficiency. Remaining structure and advantages of the second embodiment are the same as the first embodiment, and so they have the same reference numerals attached thereto, and detailed description will be omitted.

Next, a description will be given of a body temperature control device 1 of a third embodiment of the present invention, based on FIG. 5. This body temperature control device 1 is formed having a sheet shape overall, and in the form of a hat.

The body temperature control device 1 of the third embodiment is mainly used by covering the head 3 of a patient. In this way, since the body temperature control device 1 and the head 3 are closely fitted together, it is possible to carry out temperature control for the head 3 efficiently. In particular, cooling of the head 3 is effective for the following reasons. The head (1) is supplied with a lot of blood, (2) has low vasoconstrictivity, (3) has the brain as a subject organ which is close to the body temperature control device 1, and (4) has low susceptibility to tremor, which means that by controlling the temperature of this area, it is possible to control the overall body temperature more powerfully efficiently and than by controlling temperature of the whole body on a mat or the like. Also, when the body temperature control device 1 is made in a hat shape, there is the advantage that it is possible to expose the eyes, nose and mouth to the outside, which is necessary when carrying out diagnosis and treatment often. The remaining structure and advantages of the third embodiment are the same as the first embodiment, and so the same reference numerals are attached and detailed description is omitted.

In each of the embodiments described above, the body temperature control device 1 is constituted of woven fabric, but this is not limiting, and it is also possible to have a non woven fabric, where, for example closely packed hollow fiber 10 is fixed to a sheet-like laminate using a flexible resin. What is important is that the structure is such that the hollow fiber 10 makes a substantial contribution to heat exchange. If the body temperature control device 1 is made of a non-woven fabric, it is likely that the contact area between the hollow fiber 10 and the body will be further improved compared to the case of a woven fabric.

The above described embodiment are merely illustrative example, and are not intended to limit the scope of the present invention. None of the parts of the present invention are particularly limited as long as there is no departure from the spirit and scope of the present invention. For example, it is possible for the body temperature control device 1 to be thermal underwear or thermal muffler wrapped around the neck.

### INDUSTRIAL APPLICABILITY

According to the present invention, it is possible to provide a body temperature control device and a body temperature controller using such a device that are capable of efficiently controlling body temperature.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A body temperature control device comprising a hollow fiber, wherein heat transfer medium is passed through the inside of the hollow fiber.

2. The body temperature control device of claim 1, wherein the heat transfer medium is a fluid.

3. The body temperature control device of claim 1, formed in a substantially sheet shape.

4. The body temperature control device of claim 1, being flexible.

5. The body temperature control device of claim 3, being formed in a hat shape.

6. The body temperature control device of claim 4, being formed in a hat shape.

7. The body temperature control device of claim 1, wherein the hollow fiber is woven into one of either weft fibers or warp fibers.

8. The body temperature control device of claim 7, having flexible contact between the hollow fiber and the other one of the weft fibers or the warp fibers.

9. The body temperature control device of claim 1, wherein concave sections and convex sections are formed on an inner surface of the hollow fiber.

10. The body temperature control device of claim 2, wherein concave sections and convex sections are formed on an inner surface of the hollow fiber.

11. The body temperature control device of claim 1, wherein the hollow fiber is hydrophobic.

12. The body temperature control device of claim 2, wherein the hollow fiber is hydrophobic.

13. The body temperature control device of claim 3, wherein the hollow fiber is hydrophobic.

14. The body temperature control device of claim 1, wherein a heat insulating material is attached to a surface of the body temperature control device opposite to a patient.

15. A body temperature controller, comprising the body temperature control device of any one of claim 1 to claim 14, and a temperature control body for feeding temperature transfer medium to the inside of the hollow fiber of the body temperature control device.

16. A body temperature control method, comprising the steps of:
bringing the body temperature control device of any one of claim 1 to claim 14 close to a patient; and
supplying heat transfer medium to the body temperature control device.
